(19) United States
(12) Patent Application Publication
(10) Pub. No.: US 2025/0194309 A1
(43) Pub. Date: Jun. 12, 2025

(54) LIGHT SOURCE MODULE

(71) Applicant: TOYODA GOSEI CO., LTD., Kiyosu-shi (JP)

(72) Inventors: Aya KAWAOKA, Kiyosu-shi (JP); Takeshi MATSUSHIMA, Kiyosu-shi (JP); Hisayuki MIKI, Kiyosu-shi (JP)

(21) Appl. No.: 18/958,841

(22) Filed: Nov. 25, 2024

(30) Foreign Application Priority Data
Dec. 6, 2023 (JP) 2023-206416

(51) Int. Cl. H01L 33/56; A61L 2/10; H01L 33/02

(52) U.S. Cl. CPC H10H 20/854; A61L 2/10; H10H 20/8215; A61L 2202/11

(57) ABSTRACT

Provided a light source module in which ultraviolet light extraction efficiency is enhanced and stress that occurs to a light emitting element is reduced even if an area ratio of the light emitting element to a mounting substrate is large. The light source module includes a plurality of light emitting elements formed of a semiconductor and configured to emit ultraviolet light to sterilize a fluid, a mounting substrate having the plurality of light emitting elements mounted thereon, a light transmitting member transmitting the ultraviolet light and separating the plurality of light emitting elements and the mounting substrate from the fluid, and a liquid substance filled in a gap surrounded by an outer surface of the plurality of light emitting elements, the mounting substrate, and the light transmitting member.

### CROSS-REFERENCE

This application claims priority to Japanese patent application No. 2023-206416 filed on Dec. 6, 2023, the contents of which are fully incorporated herein by reference.

### BACKGROUND OF THE INVENTION

### Technical Field

The present disclosure relates to a light source module.

### Background Art

Sterilization devices to kill bacteria and viruses in running water by irradiating ultraviolet light are known. Mercury lamps have been widely used as a source of ultraviolet light. Because of using mercury, mercury lamps are highly toxic and problematically have large environmental burdens. In addition, by using mercury lamps, the size of the sterilization device is also problematically made larger. Therefore, mercury lamps are being replaced by ultraviolet LEDs.

JP-A-2021-41382 discloses a water disinfection device using an LED that emits ultraviolet light. In this disinfection device, an ultraviolet light irradiation module is disposed near an inlet provided at one end of a flow pipe through which water is distributed, and ultraviolet light is output from the ultraviolet light irradiation module in the axial direction of the flow pipe.

The ultraviolet light irradiation module includes a plurality of light emitting elements, a substrate, a housing, and a transparent window. The plurality of light emitting elements are LEDs that emit ultraviolet light and are mounted on the substrate. The plurality of light emitting elements and the substrate are housed in the housing. The housing has an opening for extracting ultraviolet light emitted from the light emitting elements. A transparent window is placed to block the opening of the housing. Thus, the light emitting elements are hermetically sealed in the ultraviolet light irradiation module.

JP-A-2016-29739 discloses a chip-on-board LED module in which a plurality of light emitting elements (LEDs) are mounted on a substrate. In this module, the plurality of light emitting elements and the substrate are protected by being covered with a transparent cured resin.

### SUMMARY OF THE INVENTION

In JP-A-2021-41382, if the ultraviolet light emitted from the light emitting element diffuses in a space of the housing, the amount of ultraviolet light to be extracted from the opening will be reduced. For this reason, by directing the ultraviolet light emitted from the light emitting element toward the opening as much as possible, the ultraviolet light extraction efficiency can be increased.

In this regard, JP-A-2021-41382 has no detailed reference to the space of the housing, i.e., the gap surrounded by the substrate, the plurality of light emitting elements, and the transparent window. It is understood that the gap merely contains air.

In JP-A-2106-29739, when a resin hardens in the manufacturing step of a chip-on-board LED module, stress due to resin hardening occurs to the light emitting element. This causes a decrease in durability of the light emitting element. In particular, when increasing an area ratio of light emitting elements to mounting substrate, a distance between adjacent light emitting elements is narrowed. However, when the distance between adjacent light emitting elements is narrowed, the resin hardens in the narrow gap. This further increases the stress that occurs to the light emitting elements.

The present disclosure is made in view of such background, and intends to provide a light source module in which the ultraviolet light extraction efficiency is improved and the stress that occurs to the light emitting elements is minimized even when the area ratio of the light emitting element to the mounting substrate is large.

An aspect of the present disclosure is a light source module including
a plurality of light emitting elements formed of a semiconductor and configured to emit ultraviolet light to sterilize a fluid;
a mounting substrate having the plurality of light emitting elements mounted thereon;
a light transmitting member transmitting the ultraviolet light and separating the plurality of light emitting elements and the mounting substrate from the fluid; and
a liquid substance filled in a gap surrounded by an outer surface of the plurality of light emitting elements, the mounting substrate, and the light transmitting member.

In the light source module of the above aspect, the liquid substance filled in the gap surrounded by the outer surface of the plurality of light emitting elements, the mounting substrate, and the light transmitting member improves the ultraviolet light extraction efficiency. The gap surrounded by the outer surface of the plurality of light emitting elements, the mounting substrate, and the light transmitting member is filled with the liquid substance. Therefore, compared to the case where the hardening resin is disposed in the gap, the stress that occurs to the light emitting elements is reduced even if the area ratio of the light emitting elements to the mounting substrate is increased.

### BRIEF DESCRIPTION OF THE DRAWINGS

FIG. 1 is a cross-sectional view of a fluid sterilization device in accordance with a first embodiment, viewed in the plane including the axis of a channel tube.

FIG. 2 is an exploded view of the fluid sterilization device in accordance with the first embodiment.

FIG. 3 is a cross-sectional view of a light source module in accordance with the first embodiment, viewed in the plane including the axis of the channel tube.

FIG. 4 is an enlarged cross-sectional view of the light source module in accordance with the first embodiment, viewed in the plane including the axis of the channel tube.

FIG. 5 is an enlarged cross-sectional view of the light source module in accordance with a second embodiment, viewed in the plane including the axis of the channel tube.

FIG. 6 is a cross-sectional view of the fluid sterilization device in accordance with a third embodiment, viewed in the plane including the axis of the channel tube.

FIGS. 7(a)-(b) is a cross-sectional view of the channel tube in an inlet, in a direction perpendicular to the central axis of the channel tube.

FIG. 8 is a schematic view of fluid flow near the inlet.

FIG. 9 is a schematic view of a light source module of the fluid sterilization device in accordance with the third embodiment.

FIG. 10 is a schematic view of a light source module of the fluid sterilization device in a fourth embodiment.

### DETAILED DESCRIPTION OF PREFERRED EMBODIMENTS

A Light source module includes a plurality of light emitting elements formed of a semiconductor and configured to emit ultraviolet light to sterilize a fluid, a mounting substrate on which the plurality of light emitting elements are directly mounted, a light transmitting member transmitting the ultraviolet light and separating the plurality of light emitting elements and the mounting substrate from the fluid, and a liquid substance filled in a gap surrounded by an outer surface of the plurality of The liquid substance filled into the gap surrounded by the outer surface of the plurality of light emitting elements, the mounting substrate, and the light transmitting member.

In the above light source module, adjacent side surfaces of adjacent light emitting elements among the plurality of light emitting elements may be in contact. According to this configuration, stresses that occur to the adjacent side surfaces can be reduced.

In the above light source module, gas may exist between adjacent side surfaces. According to this configuration, the emission of ultraviolet light from adjacent side surfaces can be suppressed and the directivity of ultraviolet light can be improved.

In the above light source module, gas may exist between the adjacent side surfaces, and a surface roughness Ra of the side surfaces may be 0.1 nm or more and 100,000 nm or less. According to these configurations, emission of ultraviolet light from the adjacent side surfaces can be curtailed and directivity of the ultraviolet light can be improved.

In the above light source module, gas may exist between the adjacent side surfaces, and the maximum distance between the adjacent side surfaces may be 200,000 nm or less. According to these configurations, the gas is made easily exist between the adjacent side surfaces. Thus, the emission of ultraviolet light from adjacent side surfaces can be curtailed and the directivity of ultraviolet light can be improved.

In the above light source module, gas may exist between the adjacent side surfaces and a contact angle of the liquid substance may be 10° or more. According to these configurations, the gas is made easily exist between the adjacent side surfaces.

In the above light source module, a distance between adjacent side surfaces of light emitting elements adjacent to each other among the plurality of light emitting elements is 5.0 mm or less. According to this configuration, ultraviolet light emitted from the side surfaces of the light emitting elements can be output from the light transmitting member. Thus, extraction efficiency in extracting ultraviolet light from the light transmitting member can be improved.

In the above light source module, when the adjacent side surfaces are separated from each other by a distance of 5.0 mm or less, the liquid substance may be filled between the adjacent side surfaces. According to this configuration, ultraviolet light can be emitted from the adjacent side surfaces. Thus, extraction efficiency in extracting ultraviolet light output from the light transmitting member can be improved

In the above light source module, when adjacent side surfaces are separated from each other by a distance of 5.0 mm or less, gas may exist between the adjacent side surfaces. According to this configuration, the radiation of ultraviolet light from the adjacent side surfaces can be curtailed and the directivity of ultraviolet light can be improved.

In the above light source module, a contact angle of the liquid substance may be 50° or less. According to this configuration, the liquid substance is more easily filled between the adjacent side surfaces.

In the above light source module, the liquid substance may be a fluorine-based inert liquid. According to this configuration, extraction efficiency in extracting ultraviolet light emitted by the plurality of light emitting elements can be effectively improved.

### First Embodiment

### 1. General Description of Fluid Sterilization Device 1

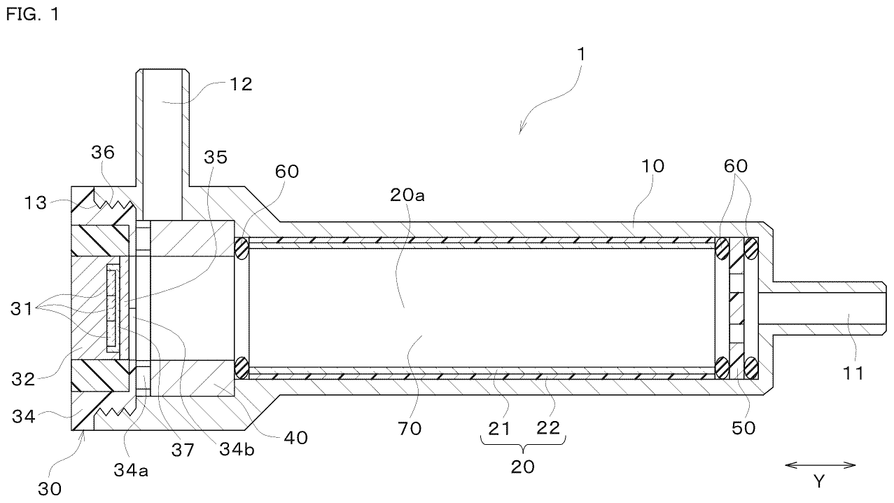

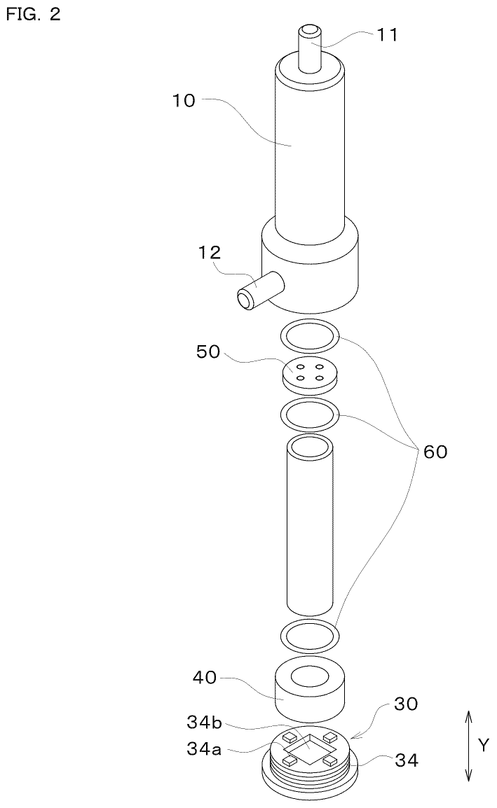

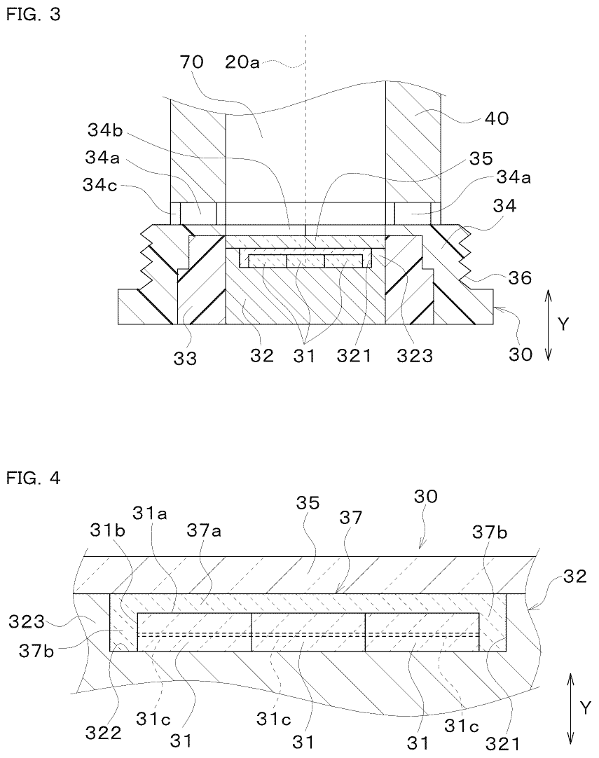

FIG. 1 is a cross-sectional view of the fluid sterilization device 1 in the first embodiment, showing a cross section in a plane including the axis of the fluid sterilization device 1. FIG. 2 is an exploded view of the fluid sterilization device 1 in the first embodiment. FIG. 3 is a cross-sectional view showing a light source module 30 in the first embodiment. As shown in FIG. 1 and FIG. 2, the fluid sterilization device 1 in the first embodiment includes a housing 10, a channel tube 20, a light source module 30, a spacer 40, and a plate 50. As shown in FIG. 2 and FIG. 3, the light source module 30 includes a light emitting element 31 (LED chip), a mounting substrate 32, a sealing member 33, a light source case 34, and a light transmitting member 35.

In the fluid sterilization device 1, fluid flows through the inside of the channel tube and the spacer 40 and is irradiated with ultraviolet light output from the light source module 30 to thereby be sterilized. The interior spaces of the channel tube 20 and the spacer form a channel space 70 (the space through which the fluid flows during sterilization and the region to which ultraviolet light is emitted). The fluid targeted for sterilization can be any liquid, for example, water, oil, alcohol, and so on. Any mixture in the state that a solid substance is mixed with a liquid can also be subject to fluid sterilization as long as the mixture has fluidity. In this specification, a fluid is defined to include a mixture of a liquid and a solid substance with fluidity. In the first embodiment, water is used as a fluid to be sterilized. Although in the first embodiment, the fluid sterilization device 1 has a substantially cylindrical shape, any shape is applicable as long as the fluid can be passed through the device. For example, any cylindrical shape such as a square cylinder can be applied.

### 2. Each Component of the Fluid Sterilization Device 1

Next, each component of the fluid sterilization device 1 is described in detail.

### 2-1. Housing 10

As shown in FIG. 1 and FIG. 2, the housing 10 has a cylindrical shape. The housing 10 has an inlet 11 on one end face in the axial direction thereof and an outlet 12 on the side surface of the cylindrical-shaped housing 10 near the other end face of the housing. The material of the housing 10 is, for example, black polypropylene (PP). Inside the housing 10, the plate 50, the channel tube 20, and the spacer 40 are coaxially disposed in this order from the inlet 11 side.

The other end face of the housing 10 is open. The housing 10 has a thread 13 provided on an inner circumference thereof near the other end face. The end face of the housing 10 on the thread 13 side is sealed by the light source module 30. The light source module 30 is fitted into the housing 10 and is screwed shut by the thread 13.

Between the housing 10 and the plate 50, between the plate 50 and the channel tube 20, and between the channel tube 20 and the spacer 40, O-rings 60 are provided respectively to thereby prevent water from leaking from the space between these components. Water flowing in from the inlet 11 is irradiated with ultraviolet light output from the light source module 30 in the course of passing through the plate 50, the channel tube 20, and the spacer in sequence, and is then discharged through the outlet 12. The positions of the inlet 11 and outlet 12 may be arbitrary, and the positions of the two may be interchanged.

### 2-2. Channel Tube 20

The channel tube 20 is formed by a cylindrical member, and the interior space of the cylindrical member forms the channel space 70. In the first embodiment, as shown in FIG. 1, the channel tube 20 has a cylindrical shape. The channel tube 20 includes a transparent tube 21 and a reflective body 22 provided in contact with the outer circumference of the transparent tube 21. The outer circumference of the reflective body 22 is in contact with the inner circumference of the housing 10. The reflective body 22 has a high reflectance of ultraviolet light. By reflecting ultraviolet light on the inner circumferential surface of the reflective body 22, ultraviolet light can be efficiently irradiated to the water flowing through the channel tube 20. Note that an extending direction of a central axis 20a of the channel tube 20 is defined as an axial direction Y.

The transparent tube 21 is made of quartz and has a cylindrical shape. Water, the object of sterilization flows through the inside of the transparent tube 21. The material of the transparent tube 21 is not limited to quartz, and any material that transmits ultraviolet light with a low absorption rate may be used. For example, sapphire, ultraviolet light-transmitting glass, or fluoroplastic may be used as a material of the transparent tube 21. In particular, the material of the transparent tube 21 preferably has a small refractive index difference from water. Thus, the material of the transparent tube 21 is preferably quartz. The material of the transparent tube 21 preferably has a refractive index of 1.3 to 1.5, for example.

The transparent tube 21 may have any thickness to the extent that the tube can have a strength to withstand water pressure and ultraviolet light transmission. The thickness of the transparent tube 21 is, for example, 0.4 to 3 mm. The material and thickness of the transparent tube 21 is preferably in such a range that the transparent tube 21 has an ultraviolet light transmittance of 80% or more when ultraviolet light enters in a surface normal direction.

The inner circumferential surface of the transparent tube 21 is preferably made as smooth as possible. In the inner circumferential surface of the transparent tube 21, for example, an RMS (a root mean square height) is set to 1 μm or less. Bacteria are hardly adhered to the indented surface of the inner circumference, and the inner circumferential surface is less likely contaminated. Therefore, the decrease in sterilization efficiency can be curtailed. Furthermore, generation of air bubbles etc. can be also prevented. The inner circumferential surface of the transparent tube 21 may be made water-repellent by forming a water-repellent film, such as fluoroplastic, on the inner circumferential surface of the transparent tube 21. In this way, the inner circumferential surface of the transparent tube 21 can be prevented from being contaminated.

The reflective body 22 is provided in contact with the outer circumference of the transparent tube 21. The reflective body 22 reflects ultraviolet light transmitted through the transparent tube 21 toward outside.

The reflective body 22 is formed by wrapping a PTFE (polytetrafluoroethylene) unsintered film around the outer circumference surface of the transparent tube 21 once or more than once. In other words, the reflective body 22 is formed of a laminated body obtained by depositing the PTFE unsintered film on the transparent tube 21. The PTFE unsintered film is formed into a sheet shape, tape shape, etc. by rolling PTFE fine powder in an unsintered state. The reflective body 22 is formed by depositing the PTFE unsintered film onto the transparent tube 21. The PTFE fine powder is composed of white powder obtained by agglomerating fine particles of PTFE.

For the PTFE fine powder, for example, paste extrusion molding powder (II-1 or II-2), which is standardized in JISK6896: 1995, can be used. Paste extrusion molding powder II-1 satisfies the following properties: apparent density of 0.50±0.15 g/ml, moisture of 0.04% or less, high temperature volatile content of 0.1% or less, melting point of 327° C.±10, specific gravity of 2.13 to 2.20, tensile strength of 17.6 MPa or more, elongation of 200% or higher. Methods for measuring these properties are specified in JISK6896: 1995. Paste extrusion molding powder II-2 has the same properties as Paste extrusion molding powder II-1 except that the specific gravity is 2.18 to 2.28.

For PTFE unsintered film, an unsintered film produced using the paste extrusion powder, which is standardized in JISK6896: 1995, and containing no additives can be used. In particular, PTFE unsintered tapes standarized in JISK6885: 2005 can be used. The PTFE unsintered tapes specified in JISK6885: 2005 satisfy the following qualities: apparent density of 1.0 g/cm³ or more, tensile strength of 7.0 MPa or more, elongation of 20% or more, volatile loss of 0.5% or less, and nonflammable. Methods for measuring these qualities are specified in JISK6885: 2005.

The PTFE unsintered film has self-bonding properties because it is unsintered. Therefore, when the PTFE unsintered film is wrapped around the transparent tube 21, there is no gap between the transparent tube 21 and the PTFE unsintered film and between the deposited PTFE unsintered films. Therefore, the PTFE unsintered film can be adhered and fixed to the transparent tube 21 without using any adhesive, and the reflective body 22 can be formed easily.

Some air layers may remain in the gap between the transparent tube 21 and the PTFE unsintered film, or between the PTFE unsintered films layered each other. Even if the air layer remains, the ultraviolet light is reflective due to the refractive index difference at the interface with the air layer. The ultraviolet light passing through the air layer is reflected by the PTFE unsintered film. Therefore, the air layer does not affect the ultraviolet reflectance of the reflective body 22 so much. Therefore, when forming the reflective body 22 by wrapping the PTFE unsintered film around the transparent tube 21, it is not necessary to wrap the PTFE film so tightly that no air layer remains, and the reflective body 22 can be formed easily.

The reflective body 22 has a high reflectivity to ultraviolet light. This is due to the following reasons. The first reason is because PTFE itself is made of a material with high ultraviolet light reflectance. The second reason is the self-bonding property of the PTFE unsintered film prevents any gap between the transparent tube 21 and the PTFE unsintered film from being formed, as well as between PTFE unsintered films layered each other. The third reason is because PTFE fine powder has a fibrous shape when shear stress is applied, the particles of the PTFE unsintered film have a fibrous shape, resulting in a higher particle density.

The thickness of the reflective body 22 is preferably 0.02 mm or more. The number of windings of the PTFE unsintered reflective film is preferably set so that the reflective body 22 has such a thickness. The reflective body 22 can have a reflectivity equal to or greater than that of barium sulfate, a common reflective material. In particular, the thickness of the reflective body 22 is preferably 0.2 mm or more. Thus, the ultraviolet light reflectance can be equal to or greater than that in the case where the channel tube 20 is formed by a PTFE bulk of 8 mm thickness. There is no upper limit to the thickness of the reflective body 22, however, the thickness is preferably set to less than 2 mm because the ultraviolet reflectance saturates as the thickness increases. The thickness of the reflective body 22 may be set to thinner than the thickness of the transparent tube 21. The thickness and width of the PTFE unsintered film is arbitrary, and may have a value as specified in JISK6885: 2005.

In the first embodiment, the channel tube 20 includes a transparent tube 21 and the reflective body 22. The entire channel tube 20 may be formed of PTFE, SUS, titanium, or polyvinyl chloride to form a cylindrical shape. The channel tube 20 may be prepared by depositing a fluororesin on the inner or outer circumferential surface of a cylindrical member formed of these materials.

O-rings 60 are disposed at each end of the channel tube 20. This prevents water from entering between the outer circumferential surface of the transparent tube 21 and the inner circumferential surface of the housing 10, more specifically between the outer circumferential surface of the transparent tube 21 and the inner circumferential surface of the reflective body 22, and between the outer circumferential surface of the reflective body 22 and the inner circumferential surface of the housing 10.

In addition to the above, the reflective body 22 can be made of aluminum. For example, the reflective body 22 can be formed by wrapping a thin film of aluminum around the outer circumferential surface of the transparent tube 21 or by depositing a thin film of aluminum on the outer circumferential surface of the transparent tube 21 by vapor deposition or sputtering. In this case, the thickness of the aluminum film should be 25 μm or more.

### 2-3. Light Source Module 30

As shown in FIG. 1, FIG. 2, and FIG. 3, the light source module 30 is cylindrical and is provided with a thread 36 on its side surface (side surface of the light source case 34). As shown in FIG. 1, the thread 36 of the light source module 30 corresponds to the thread 13 of the housing 10. The light source module 30 is screwed into the open end face of the housing 10, whereby the thread 36 of the light source module 30 and the thread 13 of the housing 10 are fitted and screwed together. The light source module 30 is disposed at one end of channel tube 20 in the axial direction via the spacer 40.

The light source module 30 outputs ultraviolet light, and the ultraviolet light enters the inside of spacer 40 and channel tube 20 from one end of the spacer 40. Thus, the ultraviolet light is irradiated to the fluid flowing through the spacer 40 and the channel tube to thereby sterilize the fluid.

### 2-4. Spacer 40

As shown in FIG. 1 and FIG. 2, the spacer 40 is disposed between the channel tube and the light source module 30. The spacer 40 is a cylindrical tube made of PTFE and is disposed coaxially with the channel tube 20. The spacer 40 efficiently reflects ultraviolet light output from the light source module 30 at a large angle to the axial direction Y of the flow path tube 20. In this way, the irradiation efficiency of ultraviolet light to the fluid flowing through the channel tube 20 can be enhanced.

### 2-5. Plate 50

As shown in FIG. 1 and FIG. 2, the plate 50 is disposed between the inlet 11 of the housing 10 and the channel tube 20. The plate 50 has a disk shape and is disposed coaxially with the channel tube 20. The material of the plate 50 is composed of PTFE. By using PTFE with high ultraviolet light reflectance, ultraviolet light reaching the end surface of the channel tube 20 on the inlet 11 side is reflected and returned to the inside of the channel tube 20, thereby increasing the irradiation efficiency of the ultraviolet light to the fluid flowing through the channel tube 20. The plate 50 is provided with a plurality of through holes. Fluid flowing from the inlet 11 is dispersed by passing through the plurality of through holes in the plate 50 and then flows into the flow path tube 20.

### 3. Each Component of Light Source Module 30

As shown in FIG. 3, the light source module 30 is substantially cylindrical in shape and includes a light emitting element 31, a mounting substrate 32, a sealing member 33, a light source case 34, and a light transmitting member 35. Furthermore, as shown in FIG. 4, the light source module 30 includes a liquid substance 37.

### 3-1. Light Emitting Element 31

As shown in FIG. 3, the light emitting element 31 is formed of a semiconductor and is mounted on a bottom 322 of a recess 321 of the mounting substrate 32. The light emitting element 31 is formed of a bare chip that emits ultraviolet light. The wavelength of the ultraviolet light can be determined depending on the fluid to be passed through the channel space 70. Since the first embodiment employs water as an objective fluid, the wavelength of the ultraviolet light is preferably 250 nm to 285 nm, which has high sterilization efficiency for water. The structure of the light emitting element 31 is not limited and can be a face-up type, flip-chip type, or top and bottom conducting type. In the first embodiment, the light emitting element 31 is a flip-chip type. Although a plurality of light emitting elements 31 are provided, the number of light emitting elements 31 is not limited as long as it is two or more, and the first embodiment includes three light emitting elements 31, as shown in FIG. 3.

The light emitting element 31 has a rectangular shape. A main surface 31a of the light emitting element 31 is a surface on an opposite side of the mounting substrate 32. A side surface 31b of the light emitting element 31 includes all the side surfaces adjacent to the main surface 31a. The angle between the main surface 31a and the side surface 31b ranges from 80 to 100° over the entire circumference of the main surface 31a, and preferably 90°.

A surface of the light emitting element 31 on the side of the mounting substrate 32 is provided with an electrode pad, which is not shown in the figure. As schematically shown in FIG. 4, an active layer 31c is formed between an n-type layer and a p-type layer inside the light emitting element 31.

Inside the light emitting element 31, an element substrate, the n-type layer, the active layer 31c, and the p-type layer are deposited in this order, and ultraviolet light is emitted at the active layer 31c. In the first embodiment, the light emitting element 31 is of a flip-chip type. Therefore, the element substrate of the light emitting element 31 is disposed on the opposite side of the mounting substrate 32, and the n-type layer, the active layer 31c, and the p-type layer are formed on the side closer to the mounting substrate 32 side than the element substrate.

The thickness of the light emitting element 31 is 100 μm or more, preferably 400 μm or more. In particular, the thickness of the element substrate of the light emitting element 31 is 100 μm or more.

Among the plurality of light emitting elements 31, adjacent side surfaces 31b contact with each other.

### 3-2. Mounting Substrate 32

The mounting substrate 32 has the light emitting element 31 mounted thereon. As shown in FIG. 3, the mounting substrate 32 has a bottomed cylindrical shape. The mounting substrate 32 includes a recess 321 on the side facing the channel tube 20. The bottom 322 of the recess 321 is planar and orthogonal to the axial direction Y.

As shown in FIG. 3, a plurality of the light emitting elements 31 are arranged on the bottom 322 of the recess 321.

As shown in FIG. 3, a side wall 323 of the recess 321 has a circular shape standing in the axial direction Y. Thus, the mounting substrate 32 has a bottomed cylindrical shape and a cylindrical space is formed inside the recess 321. The upper surface of the side wall 323 is made planer, and the light transmitting member 35 is attached thereto.

Circuit forming components (not shown) electrically connected to the light emitting element 31 is mounted on the mounting substrate 32. The circuit forming components include a drive circuit for making the light emitting element 31 emit light and a connector header that connects the drive circuit to a power cable. In addition, other electronic components, not shown, in the figures, such as a thermistor for measuring the temperature of the mounting substrate, a connector header for connecting the thermistor to the power cable, and so on are provided on the mounting substrate 32.

The material of the mounting substrate is not limited. Aluminum is used in the first embodiment. A glass epoxy substrate such as FR-4 or CEM3, or a flexible substrate made of polyimide or the like may be used as the mounting substrate. Also, the mounting substrate may also be subjected to various processings such as hole drilling.

### 3-3. Light Transmitting Member 35

The light transmitting member 35 has translucency of transmitting ultraviolet light and covers the ultraviolet light emitting side of the plurality of light emitting elements 31, as shown in FIG. 3. In the first embodiment, the light transmitting member 35 is flat and is bonded to the upper surface of the side wall 323 in the mounting substrate 32 with resin or solder. Thus, the inner space of the recess 321 is sealed. The light transmitting member 35 is configured to be in contact with the channel space 70.

The light transmitting member 35 functions as a window through which ultraviolet light emitted from the light emitting elements 31 is transmitted. The ultraviolet light emitted from the plurality of light emitting elements 31 passes through the light transmitting member and enters the channel space 70. Thus, the fluid passing through the channel space 70 is sterilized by the ultraviolet light emitted from the plurality of light emitting elements 31.

The light transmitting member 35 and the light emitting elements 31 are separated by a predetermined distance, and in the first embodiment, the inner space of the recess 321 between the light transmitting member 35 and the light emitting elements 31 is filled with the liquid substance 37.

The top surface of the light transmitting member 35 needs not be entirely in contact with the channel space 70, however, the entire top surface is in contact with the channel space 70. The side surfaces of the light transmitting member 35 may also be in contact with the channel space 70.

The light transmitting member 35 is not limited to a flat plate shape, and may have a convex lens shape. If the light transmitting member 35 has a convex lens shape, the directivity of ultraviolet light output from the light transmitting member 35 becomes higher, so that the directional angle becomes narrower. Therefore, ultraviolet light can be irradiated more efficiently to the fluid passing through the channel space 70. In particular, when difference between a refractive index of the material of the light transmitting member 35 and a refractive index of the fluid passing through the channel space 70 is large, it is preferable for the light transmitting member 35 to have a convex lens shape.

The material of the light transmitting member 35 may be any material that can transmit ultraviolet light emitted from light emitting element 31, however, the material preferably has a thermal conductivity of 0.25 W/m·K or higher. This is for the purpose of enhancing heat dissipation efficiency. For example, sapphire, quartz, borosilicate glass, and fluororesin can be used. In terms of improving heat dissipation efficiency, a material with high thermal conductivity is preferable, and sapphire is used in the first embodiment. The light transmitting member 35 may be formed of a material having a thermal conductivity higher than that of the mounting substrate 32. Heat conduction from the light emitting element 31 to the light transmitting member 35 via the inner space of the recess 321 can be more efficiently performed, so that the heat dissipation efficiency can be enhanced.

The thickness of the light transmitting member 35 is preferably 0.1 mm or more. Spreading of heat becomes larger and the heat dissipation area becomes larger, thus heat dissipation to the fluid passing through the channel space 70 via the Light transmitting member 35 can be performed more efficiently. However, if the light transmitting member is made too thick, thermal resistance increases, for this reason, the area and thickness of the light transmitting member 35 is preferably set so as to obtain appropriate heat dissipation properties.

### 3-4. Light Source Case 34

As shown in FIG. 2 and FIG. 3, the light source case 34 is cylindrical in shape and holds the mounting substrate 32 inside. The material of the light source case 34 is not limited, and in the first embodiment, polypropylene (PP) is used, however, stainless steel or the like may be used instead. The top surface of the light source case 34 (on the side of the spacer 40) is provided with an opening 34b to transmit ultraviolet light from the light emitting element 31. Inside the light source case 34, the mounting substrate 32 is disposed such that the opening 34b and the light transmitting member 35 are overlapped in the axial direction Y. In the first embodiment, the top surface of the light transmitting member 35 is disposed so as to be flush with the bottom surface of the opening 34b.

The outer upper surface of the light source case 34 has a plurality of protrusions 34a. The protrusions 34a are in contact with the spacer 40. The protrusions 34a create a gap 34c between the top surface of the light source case 34 and the spacer 40, so that fluid that has passed through the channel space 70 and reached the inside of the spacer 40 flows toward the outlet port 12 through the gap 34c. The inside of the spacer 40 and the gap 34c also form part of the flow path space 70.

The thread 36 is provided on the outer circumference surface of the light source case 34. The thread 36 corresponds to the thread 13 provided on the inner circumference surface of the housing 10. The light source module 30 fitted into the housing 10 is screwed shut by engaging the thread 36 of the light source module 30 and the thread 13 of the housing 10. The light source module 30 may be fixed to the housing 10 by a method other than screw fastening.

### 3-5. Sealing Member 33

The sealing member 33 is made of a caulking material formed to fill the gap between the mounting substrate 32 and the light source case 34. The sealing member 33 is made of a waterproof polymeric material such as urethane resin, silicone resin, or epoxy resin, for example. The sealing member 33 covers the side surfaces of the light source case 34 and circuit forming components such as a drive circuit of the mounting substrate 32 so that electronic components mounted on the mounting substrate 32 do not come into contact with water. It is noted that the top surface of the light transmitting member 35 is not covered by the sealing member 33 and is in contact with channel space 70.

It is noted that the sealing member 33 may be mixed with thermally conductive particles or thermally conductive fibers. By providing high thermal conductivity to the sealing member 33, heat from the light emitting element 31 is made to disperse easily through the sealing member 33, thereby enhancing heat dissipation. In particular, it is preferable that the light source case 34 be also made of a material with high thermal conductivity, such as metal, and that both the sealing member 33 and the light source case 34 be made of a material with high thermal conductivity.

Because the light source case 34 is in contact with the channel space 70, the light source case 34 is in direct contact with the fluid passing through the channel space 70 and can dissipate heat from the seal member 33 to the fluid passing through the channel space 70 via the light source case 34. Therefore, heat dissipation can be further enhanced. The sealing member 33 may be in contact with the channel space 70 to thereby dissipate heat from the sealing member 33 to the fluid passing through the channel space 70.

### 3-6. Liquid Substance 37

The liquid substance 37 is filled in the gap surrounded by the outer surface of the plurality of light emitting elements 31, the mounting substrate 32, and the light transmitting member 35. As shown in FIG. 4, the liquid substance 37 is filled in a main facing space 37a and a sub-facing space 37b. The main facing space 37a is a part of the gap, where the light transmitting member 35 and the main surface 31a face each other. Accordingly, the liquid substance 37 filled in the main facing space 37a is in contact with the light transmitting member 35 and the main surface 31a of the light emitting element 31.

The sub facing space 37b is a part of the gap, where the side wall 323 of the recess 321 and the side surface 31b of the light emitting element 31 face each other. Accordingly, the liquid substance 37 filled in the sub facing space 37b is in contact with the side wall 323 of the recess 321 and the side surface 31b of the light emitting element 31.

The sub facing space 37b is filled with the liquid substance 37 up to a part facing the side surface of the active layer 31c of the light emitting element 31. In other words, the side surface of the active layer 31c of the light emitting element 31 faces the liquid substance 37.

The liquid substance 37 is liquid at normal temperature and pressure. The liquid substance 37 is an inert compound and can be composed of, for example, at least one selected from the group of fluorinated inert liquids, silicon compounds, phosphate compounds, etc.

A fluorinated inert liquid as an example of the liquid substance 37 is preferably, for example, a carbon fluoride compound, which is a polymer having CF bonds. The number of carbon atoms in the carbon fluoride compound is 1.9 times the number of fluorine atoms in the carbon fluoride compound or less. Examples of the carbon fluoride compounds include, for example, perfluoropolyether (PFPE), hydrofluoroether (HFE), and so on.

### 4. Liquid Substance 37

The liquid substance 37 contains at least one selected from the group of, for example, fluoride compounds, silicon compounds, phosphate compounds, and the like. The viscosity of the liquid substance 37 is from 0.01 to 50 Pa s, preferably from 0.1 to 50 Pa s, more preferably from 1 to 50 Pa s.

A contact angle of the liquid substance 37 at a temperature of 25° C. is 15° to 25°, preferably 27° to 23°, more preferably 20°. Thus, the liquid substance 37 can be held in the main facing space 37a by capillary action. Furthermore, in the sub-facing space 37b, the liquid substance 37 is held by capillary action.

In particular, an arithmetic mean roughness Ra of the main surface 31a and the side surface 31b of the light emitting element 31 is 0.1 to 100,000 nm. This also means that the liquid substance 37 is held in the main facing space 37a and the sub-facing space 37b by capillary action. Furthermore, the light emitting element 31 is formed in a shape having no chips, recesses, or other defects in the boundary between the main surface 31a and the side surface 31b over the entire circumference of the main surface 31a. Thus, the liquid substance 37 is held in the main facing space 37a and the sub-facing space 37b. As a result, the ultraviolet light emitted from the light emitting element 31 is output from the light transmitting member 35 through the liquid substance 37.

The maximum distance between the adjacent side surfaces 31b is 200,000 nm or less. Thus, ultraviolet light emitted from adjacent side surfaces 31b can be directed toward the light transmitting member 35. Thus, ultraviolet light can be output from the light transmitting member 35.

Because the maximum distance is 200,000 nm or less, gas exists between the adjacent side surfaces 31b. Thus, emission of the ultraviolet light from the adjacent side surfaces 31b can be curtailed and the directivity of ultraviolet light is improved.

A boiling point of the liquid substance 37 is 150° C. or higher, preferably 200° C. or higher. A junction temperature Tj of the light emitting element 31 during operation is set to be 150° C. or less, preferably 100° C. or less. In particular, the boiling point of the liquid substance 37 is higher than the junction temperature Tj of the light emitting element 31 during operation by at least 50° C., preferably by at least 100° C. Therefore, even if the temperature of the light emitting element 31 rises, the liquid substance 37 can be prevented from vaporizing, and the condition in which the liquid substance 37 is held in the main facing space 37a and the sub facing space 37b by capillary action, can be maintained.

In the liquid substance 37, a transmittance of ultraviolet light of which an emission wavelength is 280 nm is 50% or more, preferably 80% or more. Furthermore, the refractive index of the liquid substance 37 is closer to that of the light transmitting member 35 than to that of air. In this embodiment, the refractive index of the liquid substance 37 is preferably larger than the refractive index of air and equal to or less than the refractive index of the light transmitting member 35. When the liquid substance 37 is a carbon fluoride compound, the refractive index of the carbon fluoride compound is, for example, between 1.2 and 1.6.

### 5. Path of Ultraviolet Light

The path of ultraviolet light emitted by the light emitting element 31 will be described. Ultraviolet light emitted at the active layer 31c of the light emitting element 31 is radiated from the main surface 31a of the light emitting element 31. The main facing space 37a between the main surface 31a of the light emitting element 31 and the light transmitting member 35 is filled with the liquid substance 37. Accordingly, the light transmitting member 35 is disposed in a direction vertical to the main surface 31a through the liquid substance 37. Ultraviolet light emitted from the main surface 31a enters the light transmitting member 35 through the liquid substance 37 and is subsequently output from the light transmitting member 35 to the outside.

Here, the main surface 31a is in contact with the liquid substance 37, and the liquid substance 37 is in contact with the light transmitting member 35. Therefore, the ultraviolet light emitted from the active layer 31c is prevented from being reflected on the main surface 31a. As a result, the ultraviolet light is emitted from the main surface 31a and then output from the light transmitting member 35.

Ultraviolet light emitted at the active layer 31c of the light emitting element 31 is also emitted from the side surface 31b of the light emitting element 31. The sub facing space 37b between the side surface 31b of the light emitting element 31 and the side wall 323 of the recess 321 is filled with the liquid substance 37. The ultraviolet light emitted from the side surface 31b is output from the light transmitting member 35 through the liquid substance 37 disposed in the sub facing space 37b.

Here, the side surface 31b is in contact with the liquid substance 37, and the liquid substance 37 is in contact with the light transmitting member 35. Therefore, the ultraviolet light emitted at the active layer 31c is prevented from being reflected at the side surface 31b toward the interior of the light emitting element 31. Therefore, the ultraviolet light is emitted from the side surface 31b.

### 6. Method of Manufacturing Light Source Module 30

The manufacturing method of the light source module 30 will be described. In a first step, each light emitting element 31 is bonded to the bottom 322 of the recess 321 of the mounting substrate 32.

Then, in a second step, the liquid substance 37 is dropped onto the main surface 31a of each light emitting element 31 and its surroundings. The volume of the liquid substance 37 to be dropped is the same as the total capacity of the main facing space 37a and the sub facing space 37b. In detail, the total volume of the liquid substance 37 to be dropped is slightly larger than the total capacity of the main facing space 37a and the sub facing space 37b.

Then, in a third step, the light transmitting member 35 is put on and bonded to the mounting substrate 32. At this time, as the main surface 31a and the light transmitting member 35 come close each other, the liquid substance 37 dripped onto the main surface 31a gradually moves to the side surface 31b after contacting the light transmitting member 35. The liquid substance 37 that has moved to the side surface 31b is held in the sub facing space 37b due to capillary action between the side surface 31b and the side wall 323 of the concave portion 321. Thus, the light source module 30 shown in FIG. 1 is completed.

In particular, because the contact angle of the liquid substance 37 dropped on the main surface 31a at a temperature of 25° C. fall within the above-mentioned angle range, the liquid substance 37 can be maintained in a state of being held in the main facing space 37a. Furthermore, because the contact angle of the liquid substance 37 to the side surface 31b at a temperature of 25° C. falls within the above-mentioned angle range, the liquid substance 37 can be maintained in a state of being held in the sub facing space 37b.

### 7. Operational Advantages

Next, the operational advantages in the light source module 30 of the first embodiment will be described in detail. According to the light source module 30, the liquid substance 37 is filled into the gap surrounded by the outer surface of the plurality of light emitting elements 31, the mounting substrate 32, and the light transmitting member 35. Accordingly, the extraction efficiency in extracting ultraviolet light emitted by the plurality of light emitting elements 31 is enhanced. The gap surrounded by the outer surface of the plurality of light emitting elements 31, the mounting substrate 32, and the light transmitting member 35 is filled with the liquid substance 37. Therefore, compared to the case where hardened resin is disposed in the gap, the stress that occurs to the light emitting elements 31 is reduced even if the area ratio of the light emitting elements 31 to the mounting substrate 32 is large.

In the light source module 30, the adjacent side surfaces 31b contact with each other. According to this configuration, the stresses that occur to the adjacent side surfaces 31b can be reduced.

In the light source module 30, gas exists between the adjacent side surfaces 31b. Thus, emission of the ultraviolet light from the adjacent side surfaces 31b is curtailed and directivity of the ultraviolet light is improved.

In the light source module 30, the surface roughness Ra of the side surfaces 31b is between 0.1 nm and 100,000 nm. According to this configuration, emission of the ultraviolet light from the adjacent side surfaces 31b can be curtailed and directivity of the ultraviolet light can be improved.

In the light source module 30, the maximum distance between the adjacent side surfaces 31b is 200,000 nm or less. Thus, gas easily exists between the adjacent side surfaces 31b. Therefore, emission of ultraviolet light from the adjacent side surfaces 31b can be curtailed and directivity of the ultraviolet light can be improved.

In the light source module 30, because the contact angle of the liquid substance 37 is 10° or more, gas easily exists between the adjacent side surfaces 31b. Therefore, emission of ultraviolet light from the adjacent side surfaces 31b can be curtailed and directivity of the ultraviolet light can be improved.

In the light source module 30, the liquid substance 37 is a fluorinated inert liquid. Thus, the extraction efficiency in extracting the ultraviolet light emitted by the plurality of light emitting elements 31 can be effectively enhanced.

### Second Embodiment

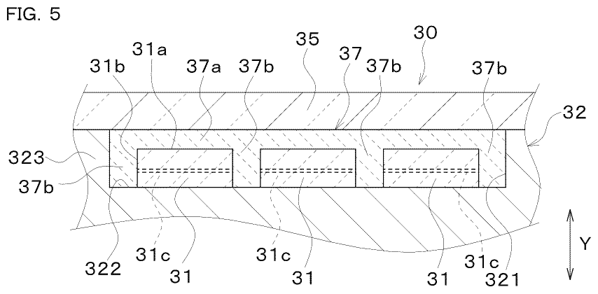

In the first embodiment, among the plurality of light emitting elements 31, the adjacent side surfaces 31b are in contact with each other. In the second embodiment, as shown in FIG. 5, among the plurality of light emitting elements 31, adjacent side surfaces 31b are separated by a predetermined distance.

The adjacent side surfaces 31b are separated from each other by a distance of 5.0 mm or less. The contact angle of the liquid substance 37 is 50° or less.

According to this configuration, the liquid substance 37 is filled between the adjacent side surfaces 31b. Furthermore, the liquid substance 37 can be maintained in a state of being held between adjacent side surfaces 31b.

A gap where the adjacent side surfaces 31b face with each other is filled with the liquid substance 37. Therefore, the ultraviolet light emitted at the active layer 31c of the light emitting element 31 and irradiated from the mutually adjacent side surfaces 31b is output from the light transmitting member 35 through the liquid substance 37.

Here, the adjacent side surfaces 31b are in contact with the liquid substance 37 and the liquid substance 37 is in contact with the light transmitting member 35. Therefore, the emitted ultraviolet light is prevented from being reflected at the side surfaces 31b and is output from the light transmitting member 35.

In the light source module 30 according to the second embodiment, the adjacent side surfaces 31b are separated from each other by a distance of 5.0 mm or less. According to this configuration, the ultraviolet light irradiated from the adjacent side surfaces 31b can be output from the light transmitting member 35. Thus, the extraction efficiency in extracting ultraviolet light from the light transmitting member 35 can be enhanced.

In the light source module 30 according to the second embodiment, the space between the adjacent side surfaces 31b is filled with the liquid substance 37. Therefore, ultraviolet light can be irradiated from the adjacent side surfaces 31b. Thus, the extraction efficiency in extracting the ultraviolet light output from the light transmitting member 35 can be enhanced.

In the light source module 30 according to the second embodiment, a contact angle of the liquid substance 37 is 50° or less, thus, the liquid substance 37 is easily filled between the adjacent side surfaces 31b.

### Modification of the Second Embodiment

In the second embodiment, the liquid substance 37 is filled between the adjacent side surfaces 31b. However, gas (e.g., air) may exist at least partially between the adjacent side surfaces 31b. Gas may exist entirely between the adjacent side surfaces 31b. The gas may exist only partially between the adjacent side surfaces 31b. In other words, the gas and the liquid substance 37 may exist mixedly between the adjacent side surfaces 31b. According to this configuration, radiation of ultraviolet light from the adjacent side surfaces 31b may be curtailed and directivity of the ultraviolet light may be improved.

### Third Embodiment

### 1.1 General Description of Fluid Sterilization Equipment 2

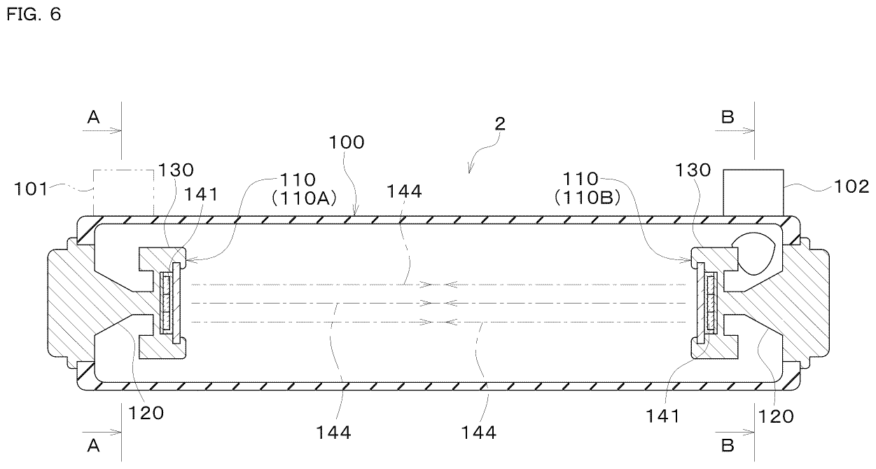

FIG. 6 is a schematic view that shows the fluid sterilization device 2 in the third embodiment. As shown in FIG. 6, the fluid sterilization device 2 in the third embodiment includes a channel tube 100 and a light source module 110. The light source module 110 includes a light emitting element 141, a column 120, and a storage part 130.

In the fluid sterilization device 2, a fluid is supplied to the channel space inside the channel tube 100 from an inlet 101 of the channel tube 100. The fluid is irradiated with ultraviolet light output from the light source module 110, and thus is sterilized. The sterilized fluid is then discharged from an outlet 102. A fluid to be sterilized can be gas or a liquid, a mixture of gas and liquid, a mixture of gas and powdered solid, and so on, as long as it has flowability. In the case of a liquid, the fluid to be sterilized may be, for example, water, oil, alcohol, or a dissolved solution using these as solvents.

### 2. Each Component of Fluid Sterilization Device 2

Next, each component of the fluid sterilization device 2 is described in detail.

### 2-1. Channel Tube 100

The channel tube 100 has a cylindrical shape and has a cylindrical space inside. This space is a channel space through which the fluid to be sterilized flows. The materials of the channel tube 100 are metals such as stainless steel, titanium, aluminum, and iron, resin materials such as polyvinyl chloride, polyethylene and polytetrafluoroethylene (PTFE), and glass such as quartz glass.

Stainless steel, titanium, iron, and polyvinyl chloride are high in absorption of ultraviolet light and low in reflectance of ultraviolet light in comparison with the materials such as aluminum and polytetrafluoroethylene (PTFE), which have high reflectance of ultraviolet light. Stainless steel and titanium have high corrosion resistance to seawater. Glass including, for example, quartz glass, soda lime glass, borosilicate glass, and lead glass, is translucent to ultraviolet light and is low in ultraviolet light reflectance. In a material with low ultraviolet light reflectance for use in the channel tube 100, for example, the ultraviolet light reflectance on the surface to be in contact with the fluid to be sterilized is 35% or less.

Both ends of the channel tube 100 are respectively provided with one light source module 110 each. An inlet 101 is provided on the peripheral wall of the channel tube 100 on one end side in the axial direction (left side in FIG. 6) of the tube. An outlet 102 is provided on the peripheral wall of the channel tube 100 on the other end side (right side in FIG. 6). In the following, the end of the channel tube 100 on the inlet 101 side is defined as a first end, and the end of the channel tube 100 on the outlet 102 side is defined as a second end.

A first light source module 110A is disposed on the inlet 101 side of the channel tube 100, and a second light source module 110B is disposed on the outlet 102 side. In the following, the first light source module 110A and the second light source module 110B are simply expressed as a light source module 110 without distinguishing between their names.

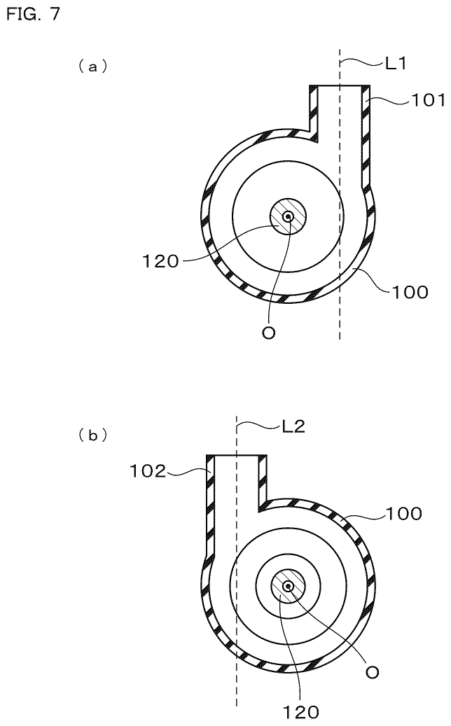

FIG. 7(a) shows the location of the inlet 101 and FIG. 7(b) shows the location of the outlet 102. FIG. 7(a) and FIG. 7(b) are cross-sectional views of the channel tube 100 cut in a plane perpendicular to the central axis of the channel tube 100 and viewed from the first end surface to the second end surface.

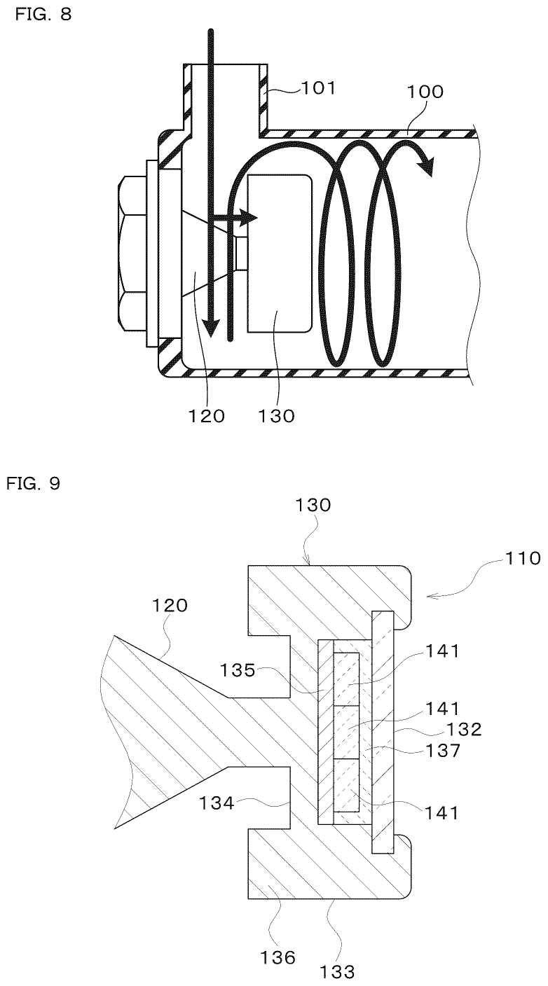

As shown in FIG. 7(a), the inlet 101 is disposed such that the direction of the fluid flowing from the inlet 101 is offset with respect to a center O of the channel tube 100. In other words, the inlet 101 is disposed offset such that a central axis L1 of does not pass through the center O of the channel tube 100. By disposing the inlet 101 in this way, a helical flow can be formed in the channel space in the channel tube 100, as shown in FIG. 8. The tangential direction of the helical flow on the central axis L1 of the inlet 101 coincides with the direction of the central axis L1 of the inlet 101.

The outlet 102 is also disposed such that the outflow direction is offset from the center O of the channel tube 100, as shown in FIG. 7(b). In other words, the outlet 102 is disposed off set such that a central axis L2 does not pass through the center O of the channel tube 100. According to this configuration, the helical flow can be maintained on the outlet 102 side, and the tangential direction of the helical flow on the central axis L2 of the outlet 102 coincides with the direction of the central axis L2 of the outlet 102.

### 2-2. Light Source Module 110

The light source module 110 includes the column 120, the storage part 130, and the light emitting elements 141. The storage part 130 houses the light emitting elements 141.

The first light source module 110A will be described below, but the second light source module 110B has substantially the same structure.

The column 120 protrudes axially from the first end of the channel tube 100 toward the second end of the channel tube 100, as shown in FIG. 6. The column 120 includes a truncated cone shaped portion. The central axis of the column 120 coincides with the central axis of the channel tube 100. In other words, the central axis of the column 120 is disposed coaxially with the central axis of the channel tube 100.

The inclination angle (angle with respect to the bottom) of the circumference of the truncated cone is, for example, 30° to 70°. In the column 120, the large diameter end is connected to the first end of the channel tube 100, and the small diameter end is connected to the storage part 130.

The column 120 is not limited to a truncated cone shape, and may have any shape as long as it is formed in a shape that tapers toward the second end. The column 120 may be formed in a shape that tapers stepwise, but preferably it is formed in a continuously tapering shape. For example, the column 120 may be formed in a truncated pyramid shape. However, the shape of the column 120 has preferably a truncated cone shape in order to form a helical flow. The entire column 120 needs not be formed in a truncated cone shape. For example, part of the column 120 may be formed in a truncated cone shape and the other part may be formed in a cylindrical shape. For example, as shown in FIG. 6, the tip of the column 120 that is connected to the storage part 130 may be formed in a cylindrical shape, and the root that is connected to the first end of the channel tube 100 may be formed in a truncated cone shape.

As shown in FIG. 9, the storage part 130 that houses the light emitting elements 140 includes a light transmitting member 132, a seat portion 133, and a mounting substrate 135.

The seat portion 133 has a bottomed cylindrical shape with an end face opened, and the bottom is connected to the end of the column 120. The central axis of the seat portion 133 is aligned with the central axis of the column 120. In other words, the central axis of the seat portion 133 is disposed coaxially with the central axis of the column 120. As mentioned above, the central axis of the column 120 is disposed coaxially with the central axis of the channel tube 100. Accordingly, the central axis of the pedestal portion 133 is also disposed on the same axis as the central axis of the channel tube 100.

The mounting substrate 135 is disposed at the bottom in the interior space of the seat portion 133, and the light emitting elements 140 is mounted on the mounting substrate 135. The light transmitting member 132 is provided on the end surface of the seat portion 133 to seal the interior space of the seat portion 133. The light transmitting member 132 is made of a material, for example, quartz or sapphire, which transmits ultraviolet light emitted from the light emitting elements 140. A photocatalytic film that transmits ultraviolet light may be formed on the outer surface of the light transmitting member 132 to thereby inhibit the growth of bacteria on the light transmitting member 132 and to prevent organic contamination. The light transmitting member 132 is not limited to a flat plate, and may have a lens shape. The light transmitting member 132 may be, for example, a TIR lens, a fly-eye lens, or a Fresnel lens.

The seat portion 133 is formed such that it extends from the tip of the column portion 120 outward in the radial direction of the column 120 over the entire circumference of the tip of the column 120. In other words, the outer diameter of the seat portion 133 is larger than the outer diameter of the tip of the column 120. Therefore, the bottom surface of the seat portion 133 is exposed to the channel space except for the part connected to the column portion 120.

A peripheral wall 136 protruding toward the first end is formed at the outer edge of the bottom of the seat portion 133. Thus, a recess 134, which is more concave than the peripheral wall 136, is formed in the portion of the bottom surface of the seat portion 133, which is surrounded by the peripheral wall 136. The recess 134 is provided to stay the fluid near the bottom of the seat portion 133 as much as possible. The fluid that stays near the bottom of the seat portion 133 can cool the seat portion 133. In other words, the light emitting elements 141, which generate heat, can be cooled through the seat portion 133.

In the third embodiment, the circumferential wall 136 is provided over the entire circumference of the bottom surface of the seat portion 133, however, it may be provided only partially in the circumferential direction. By providing the peripheral wall 136 only partially on the bottom surface of the seat portion 133 in the circumferential direction, air accumulation in the recess 134 is curtailed and cooling efficiency is enhanced.

The peripheral wall 136 is preferably located outside the light emitting elements 141, viewed in the direction along the central axis of the channel tube 100. In other words, viewed in the direction along the central axis of the channel tube 100, the recess 134 preferably overlaps the light emitting elements 141. The fluid that stays near the bottom of the seat portion 133 can cool the seat portion 133 more efficiently. In other words, the light emitting elements 141, which generate heat, can be cooled more efficiently through the seat portion 133.

In the third embodiment, the seat portion 133 is formed in a bottomed cylindrical shape, but it may have any shape as long as it is formed in a bottomed cylindrical shape. The seat portion 133 may, for example, be formed in a bottomed square cylindrical shape (polygonal box shape). However, in order to generate a helical flow, the seat portion 133 is preferably formed in a bottomed cylindrical shape, as in the third embodiment.

The column 120 and the seat portion 133 are preferably made of a metallic material with high thermal conductivity, such as aluminum. The column 120 and the seat portion 133 may be made of titanium and have a photocatalytic film by oxidizing its surface. This will inhibit the growth of bacteria on the column 120 and the seat portion 133.

The light emitting elements 141 are mounted directly on the mounting substrate 135. A plurality of the light emitting elements 141 may be mounted on one mounting substrate 135. In FIG. 6 and FIG. 9, three light emitting elements 141 are mounted.

The light emitting elements 141 are formed of a semiconductor and emits ultraviolet light. The wavelength of ultraviolet light is preferably 250 nm to 285 nm, which has high sterilization efficiency. The structure of the light emitting element 141 is not limited and can be a face-up, flip-chip, or top and bottom conducting type.

The mounting substrate 135 has the light emitting elements 141 mounted thereon. The mounting substrate 135 is formed of aluminum. The mounting substrate 135 may be a flexible substrate made of glass epoxy substrate such as FR-4 and CEM3, or polyimide.

As shown in FIG. 9, the mounting surface of the mounting substrate 135 on which the light emitting elements 141 are mounted is formed in a plane perpendicular to the central axis of the base portion 133. On the mounting surface of the mounting substrate 135, the adjacent side surfaces of the three light emitting elements 141 are in contact with each other.

On the mounting substrate 135, circuit forming components, not shown in the figure, are mounted, which are electrically connected to the light emitting elements 141. The circuit forming components include a drive circuit for emitting light from the light emitting elements 141 and a connector header for connecting the drive circuit and a power cable. As other electronic components not shown in the figure, a thermistor for measuring a temperature of the mounting substrate 135, a connector header for connecting the thermistor and the power cable, and other components are provided on the mounting substrate 135.

As shown in FIG. 9, the storage part 130 encapsulates a liquid substance 137. The liquid substance 137 is filled in the gap surrounded by the storage part 130, the outer surface of the plurality of light emitting elements 141, the mounting substrate 135, and the light transmitting member 132. The light emitting elements 141 and the liquid substance 137 are to the same as the light emitting elements 31 and the liquid substance 37 in the first embodiment. Therefore, a detailed description of the light emitting elements 141 and liquid substance 137 in the third embodiment is omitted.

### 2-3. Optical Axis 144 of Light Emitting Element 141

The optical axis 144 of the light emitting element 141 is the axis line in the direction in which the semiconductor layers for emitting ultraviolet light in the ultraviolet light emitted by the light emitting element 141 are laminated, that is, the axis line perpendicular to the main surface of the element substrate on which the semiconductor layers are laminated. The optical axis 144 is, for example, an axis line located at the center in the irradiation range of the ultraviolet light emitted by the light emitting element 141 having a specified light distribution characteristics.

The optical axis 144 may be, in some cases, an imaginary line extending from the light emitting element 141 in the direction such that light intensity of the ultraviolet light becomes maximum in the irradiation range of ultraviolet light, but is not limited thereto. In the third embodiment, the optical axis 144 of the light emitting element 141 is on a perpendicular line relative to the portion of the mounting surface where the light emitting element 141 is mounted.

As described above, the mounting surface of the mounting substrate 135 is formed in a planar shape orthogonal to the central axis of the seat portion 133. Therefore, the optical axis 144 of the light emitting element 141 takes a direction parallel to the central axis of the seat portion 133.

As described above, the central axis of the seat portion 133 takes a direction coaxially with the central axis of the channel tube 100. Therefore, the optical axis 144 of the light emitting element 141 takes a direction parallel to the central axis of the channel tube 100.

### 3. Fluid Flow Path

Next, the flow path of the fluid in the channel space is described. FIG. 8 schematically illustrates the flow path of the fluid near the first end of the channel tube 100. As shown in FIG. 8, the fluid entering the channel space inside the channel tube 100 from the inlet 101 hits the periphery of the truncated cone shaped portion of the column 120, and is reflected in the axial direction because of the inclined periphery. Thus, a flow toward the storage part 130 is formed. In this way, the flow-in fluid can be efficiently made in contact with the storage part 130, and thus the cooling efficiency of the storage part 130 can be enhanced.

Because the outer diameter of the seat portion 133 is larger than the outer diameter of the tip of the column 120, the fluid can be contacted with the bottom of the seat portion 133 (the left side surface in FIG. 8). As a result, the seat portion 133 can be cooled efficiently.

The peripheral wall 136 provided on the bottom of the seat portion 133 forms a recess 134 surrounded by the peripheral wall 136. As a result, the fluid easily stay on backside of the seat portion 133. Therefore, heat can be efficiently conducted from the bottom of the seat portion 133 to the fluid to thereby enhance the cooling efficiency of the seat portion 133.

Because the inlet 101 is offset, a flow swirling around the column section 120 is formed, as shown in FIG. 8. The column 120 has a shape that becomes thinner as going toward the second end from the first end. Therefore, the fluid flows in the direction from the first end to the second end while swirling around the column 120. Thus, a helical flow is formed in the channel space. By forming a helical flow, the staying time of the fluid in the channel space is made longer to thereby increase the time of irradiating the fluid with ultraviolet light. As a result, the sterilization efficiency can be enhanced.

In the second light source module 110B on the outlet 102 side, the column 120 has a shape that becomes thinner as going toward the first end from the second end. Therefore, fluid flowing from the first end toward the second end can be reflected in the radial direction of the channel tube 100 by the column 120. As a result, the staying time of the fluid in the vicinity of the storage part 130 on the outlet 102 side can be lengthened, and thus, the storage part 130 can be cooled efficiently.

### 4. Operational Advantage

In the same way as in the first embodiment, the storage part 130 encapsulates the liquid substance 137, thus the ultraviolet light extraction efficiency can be enhanced as in the first embodiment.

### Modification of the Third Embodiment

In the fluid sterilization device 2 according to the third embodiment, the light source module 110 is provided on the inlet 101 side and the outlet 102 side respectively, however, for example, in the case where the channel tube 100 is short, the light source module 110 may be provided only on the inlet 101 side. In this case, a reflective member that reflects ultraviolet light may be disposed on the end surface on the second end side of the channel tube 100. Irradiating the fluid with the ultraviolet light reflected by the reflective member can enhance the sterilization efficiency. The light source module 110 may be provided only on the outlet 102 side. In this case as well, the sterilization efficiency can be enhanced by providing a reflective member on the end surface on the first end side of the channel tube 100. PTFE, SUS, Ti, etc. can be used for the reflective member.

### Fourth Embodiment

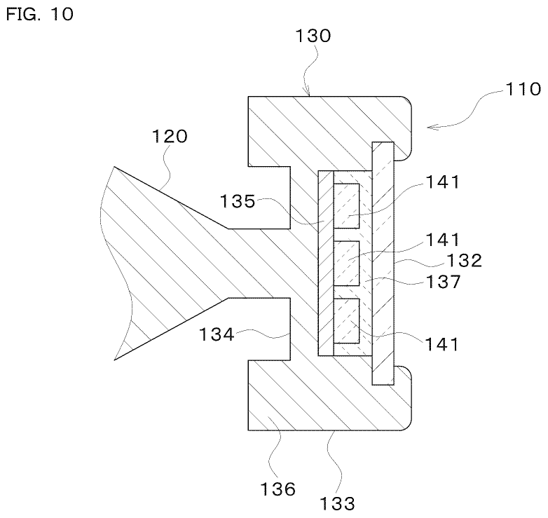

In the third embodiment, on the mounting surface of the mounting substrate 135, adjacent side surfaces of the adjacent light emitting elements 141 among the three light emitting elements 141 are in contact with each other. In contrast, in the fourth embodiment, as shown in FIG. 10, among the three light emitting elements 141, adjacent side surfaces of the adjacent light emitting elements 141 are separated from each other by a predetermined distance on the mounting surface of the mounting substrate 135.

The storage part 130 encapsulates a liquid substance 137. The liquid substance 137 is filled in the gap enclosed by the storage part 130, the outer surface of the plurality of light emitting elements 141, the mounting substrate 135, and the light transmitting member 132. The light emitting elements 141 and the liquid substance 137 are to the same as the light emitting elements 31 and the liquid substance 37 in the second embodiment. Therefore, a detailed description of the light emitting element 141 and liquid substance 137 in the fourth embodiment is omitted.

In the same way as in the second embodiment, the storage part 130 encapsulates the liquid substance 137. Furthermore, the liquid substance 137 is disposed between adjacent side surfaces of the adjacent light emitting elements 141. Therefore, as in the second embodiment, the ultraviolet light extraction efficiency can be enhanced.

### OTHER MODIFICATIONS

Although the above embodiments relate to sterilization of liquid, any fluid can be sterilized, including gas, mixtures of gas and liquid, and mixtures of gas and powdered solid.

The present disclosure is not limited to the above embodiments, but can be applied to various embodiments to the extent not departing from the gist thereof.

What is claimed is:

1. A light source module comprising:
a plurality of light emitting elements formed of a semiconductor and configured to emit ultraviolet light to sterilize a fluid;
a mounting substrate having the plurality of light emitting elements mounted thereon;
a light transmitting member transmitting the ultraviolet light and separating the plurality of light emitting elements and the mounting substrate from the fluid; and
a liquid substance filled in a gap surrounded by an outer surface of the plurality of light emitting elements, the mounting substrate, and the light transmitting member.

2. The light source module according to claim 1, wherein adjacent side surfaces of adjacent light emitting elements among the plurality of light emitting elements are in contact.

3. The light source module according to claim 2, wherein gas exists between the adjacent side surfaces.

4. The light source module according to claim 3, wherein a surface roughness Ra of the side surfaces is 0.1 nm or more and 100,000 nm or less.

5. The light source module according to claim 3, wherein a maximum distance between the adjacent side surfaces is 200,000 nm or less.

6. The light source module according to claim 3, wherein a contact angle of the liquid substance is 10° or more.

7. The light source module according to claim 1, wherein a distance between adjacent side surfaces of light emitting elements adjacent to each other among the plurality of light emitting elements is 5.0 mm or less.

8. The light source module according to claim 7, wherein the liquid substance is filled between the adjacent side surfaces.

9. The light source module according to claim 7, wherein gas exists between the adjacent side surfaces.

10. The light source module according to claim 7, wherein, a contact angle of the liquid substance is 50° or less.

11. The light source module according to claim 1, wherein the liquid substance is a fluorine-based inert liquid.
